(19) United States
(12) Patent Application Publication
(10) Pub. No.: US 2024/0083834 A1
(43) Pub. Date: Mar. 14, 2024

(54) Process for Enriching Acetic Acid From Aqueous Acetic Acid Mixtures Containing Other Lower Carboxylic Acids

(71) Applicant: proFagus GmbH, Bodenfelde (DE)

(72) Inventors: Martin Schwiderski, Northeim (DE); Bernward Wosnitza, Rosdorf (DE); Frank Höfer, Bad Dürkheim (DE)

(21) Appl. No.: 18/367,191

(22) Filed: Sep. 12, 2023

(30) Foreign Application Priority Data
Sep. 13, 2022 (DE) 10 2022 123 269.7

(51) Int. Cl. C07C 51/43

(52) U.S. Cl. CPC C07C 51/43

(57) ABSTRACT

A process for enriching acetic acid from aqueous acetic acid mixtures containing other lower carboxylic acids such as formic acid, propionic acid and butyric acid by crystallization.

### CROSS-REFERENCE TO RELATED APPLICATIONS

This application claims priority to German Patent Application No. 10 2022 123 269.7 filed Sep. 13, 2022, the disclosure of which is incorporated herein by reference in its entirety and for all purposes.

### BACKGROUND

The present disclosure relates to a process for enriching acetic acid from aqueous acetic acid mixtures containing other lower carboxylic acids such as formic acid, propionic acid and butyric acid by crystallization.

Today, acetic acid is one of the essential basic products of the chemical industry. It serves as a raw material for many other products and value chains (vinyl acetate, acetic anhydride, cellulose acetate, paints and adhesives, acetic acid esters as solvents).

There is a multitude of processes for preparing acetic acid:
Fermentation of wine
Isolation of acetic acid from the wood vinegar which is obtained during wood distillation
Oxidation of acetaldehyde (Wacker process)
Oxidation of n-butane
Carbonylation of methanol (Monsanto process)

### Wood Distillation

Wood vinegar is a dilute aqueous acetic acid solution comprising other secondary components. Turning it into an anhydrous quality by distillation can only be achieved at very great expense. For this reason, the processes for obtaining anhydrous acetic acid that are described in the first edition of Ullmann's Encyclopaedia of Industrial Chemistry from 1914 (Ullmanns Enzyklopadie der Technischen Chemie) concentrate on release from the acetate salts by mineral acids, preferably using sulfuric acid. The most important raw material was the wood vinegar obtained by dry distillation of wood and available more cost-effectively than fermentation vinegar. Reaction with lime yielded acetate of lime containing up to 83% calcium acetate. The raw acetic acid obtained therefrom by acid treatment had to be subsequently cleared of the by-products of wood pyrolysis by rectification. At the time, around 35 000 t of lime acetate were worked up to acetic acid and its derivatives annually in Germany.

### Oxidation of Acetaldehyde

Whereas the first two editions of Ullmann's merely described the oxidation of acetaldehyde to acetic acid as a possibility for future plants, this route was already the dominant one in the third edition. At that time (1953), wood vinegar was virtually irrelevant, and only around 10% was obtained (based on 100% acetic acid) after the fermentation process. Acetaldehyde is successfully oxidized with high selectivity via peracetic acid as an intermediate stage. The reaction is carried out in acetic acid at 50-70° C. in bubble column reactors, using catalysts such as manganese acetate to decompose the peracetic acid. Large plants were operated, for example, by Farbwerke Hoechst and by Celanese Corp. By the end of the Second World War, acetaldehyde was mainly prepared by the mercury-catalysed addition of water to acetylene.

Since acetylene was obtained from calcined lime and coke in electrically heated carbide furnaces via the intermediate stage calcium carbide, this was a classic example of coal-based chemistry. With the boom in petrochemistry after the Second World War, this synthesis was completely replaced by the oxidation of ethylene by the Wacker-Hoechst process. The reaction of ethylene with (atmospheric) oxygen takes place in a bubble column reactor by means of the redox catalyst CuCl₂/PdCl₂. The palladium(II) chloride oxidizes ethylene to acetaldehyde with formation of metallic palladium(0). Reoxidation thereof is achieved by copper(II) chloride, which for its part is regenerated using (atmospheric) oxygen. The preparation of acetaldehyde is also possible by the catalytic dehydrogenation of ethanol in the gas phase. In the past, this route was not economically viable owing to the excessively high costs of ethanol, but it might be relevant in the future on account of the better availability of bioethanol.

Oxidation of n-Butane

The direct oxidation of butane or naphtha to acetic acid as the main product has been known since as early as 1884. As oil refining greatly expanded, gaseous hydrocarbons were obtained in the refineries, and these were initially neither used for fuels nor used in the chemical industry. Consequently, from 1952, butane oxidation was utilized by companies such as Union Carbide Corp., Celanese Corp. and Chemische Werke Hüls in plants having acetic acid capacities of up to 300 000 t/a. The reaction takes place in the liquid phase via peroxy radicals at 130-200° C. under a pressure of 60-80 bar, using acetic acid as a solvent with complete oxygen conversion. Work-up of the reactor output is complex, since not only is acetic acid formed, but also formic acid, propionic acid and butyric acid as well as neutral products such as ketones, aldehydes, esters and alcohols. Investment costs are adversely affected by the need to use expensive, corrosion-resistant materials. Owing to the sharp increase in energy prices, this preparation route is no longer economically viable and the operation of these plants has been discontinued.

### Carbonylation of Methanol

Today, the carbonylation of methanol is the preferred route for preparing acetic acid and is used in virtually all new plants from the last 30 years. There are two reasons for this: these are the good availability and low costs of the raw materials, in addition to the very high selectivity of this synthesis. Methanol carbonylation has also gone through multiple cycles of innovation. Known in principle since 1913, the systematic work of Reppe and coworkers finally resulted in industrially usable catalysts based on metal carbonyls in 1941. However, because of the drastic reaction conditions and the corrosive reaction mixture, it was not until 1960 in Ludwigshafen that an industrial plant could be put into operation when appropriate alloys were available. This BASF high-pressure process operated at 250° C. and 700 bar with a homogeneous catalyst based on cobalt iodide. Despite this pioneering achievement, this process did not gain acceptance, even though this plant was gradually expanded to a capacity of eventually around 45 000 t/a and a second plant was operated under license. This is because just ten years after start-up of the BASF process, an acetic acid plant based on a new low-pressure process having a capacity of 135 000 t/a was put into operation by Monsanto Corp.

Advances in homogeneous catalysis with precious metal complexes formed the basis of the development of a catalyst based on soluble rhodium compounds and methyl iodide that was already highly active at 180° C. and a pressure of only 30 bar. At the same time, this catalyst achieved an impressive acetic acid selectivity of around 99% in relation to methanol and around 90% in relation to carbon monoxide. Accordingly, the low-pressure process was distinctly more cost-effective in terms of both raw material costs and fixed investment-related costs and thus gained acceptance worldwide.

The Monsanto acetic acid process is an outstanding example of the efficiency of homogeneous catalysis and is mentioned in any relevant textbook. The first licensee of the process was Celanese Chem. Comp. in a plant in Clear Lake, Texas, having a capacity of 270 000 t/a. In the early 1980s, Celanese developed the process further, which included modification of the catalyst with inorganic iodides, thereby making it possible to carry out the reaction at low water concentrations. This new process allowed the gradual expansion of the plant to three times its capacity with comparatively low investment. Following the realignment of Monsanto, the rights to this process were sold to BP Chemicals in 1986. BP also developed the low-pressure process further by use of homogeneous iridium/methyl iodine catalysts. As a result, it was likewise possible to operate the synthesis at low water concentrations with very high space-time yields.

World-scale acetic acid plants of today of the technology and world market leaders BP or Celanese have capacities of around 1 million t/a. Even though the acetic acid processes of today have reached an impressive efficiency, there is potential for further innovations. For instance, BP Chemicals recently announced the launch of a new technology based on the carbonylation of dimethyl ether to methyl acetate. According to patent literature, use is made of a non-corrosive, heterogeneous zeolite catalyst.

Undesirable by-products are formed in all processes for preparing acetic acid. The fermentation of glucose yields butyric acid and lactic acid. The oxidation of butane produces not only short-chain alcohols and esters but also formic acid and butyric acid as by-products. Formic acid is also obtained when acetaldehyde is oxidized. In the Monsanto process, ethanol impurities in the methanol mean that undesirable propionic acid is produced during carbonylation.

In all of the processes mentioned above, the contaminating components must therefore be removed from the acetic acid by suitable processes.

Distillative separation of aqueous mixtures of acetic acid and formic acid only leads to limited yields of acetic acid owing to the binary maximum-boiling azeotrope of formic acid and water and to an additionally occurring ternary azeotrope of acetic acid, formic acid and water. In addition, the close proximity of boiling points means that high operating and investment costs have to be expected for such a separation task.

U.S. Pat. No. 3,660,483A describes a process in which the formic acid is removed from acetic acid by means of reactive rectification. To this end, methanol is reacted with formic acid and the resultant methyl formate can be readily separated from acetic acid by distillation.

In U.S. Pat. No. 3,801,629A, water and formic acid are removed from an acetic acid mixture by means of reactive rectification as well. Here, n-butanol or isoamyl alcohol is used in instead of methanol.

In WO02/053524A2, a mixture of acetic acid, formic acid and water is first dewatered with diisopropyl ether, followed by distillative removal of the formic acid with the aid of cyclopentane.

### SUMMARY

All of the processes described have some disadvantages. The acetic acid vapour which occurs as a result of distillation leads to corrosion on the plant parts and thus to increased maintenance costs. Use of solvents which are highly flammable, explosive and harmful to health should be avoided for reasons of safety and health. None of the processes described separates both the low-boiling components water and formic acid, which are low-boiling compared to acetic acid, and the high-boiling components propionic acid and butyric acid in one process stage. In principle, the distillative separation tasks are associated with high equipment expenditure, high energy use and complex controls.

The present disclosure was done against the background of the above-described prior art, it being an aim to develop a process which depletes the concentrations of both the low boilers water and formic acid and the high boilers propionic acid and butyric acid in one process step (and accordingly enriches the concentration of acetic acid). The temperature shall be kept at a moderate level because of problems of corrosion. The use of solvents shall be dispensed with.

A process is disclosed herein for enriching acetic acid from aqueous acetic acid mixtures containing at least 50% by weight of acetic acid and, as secondary constituents, formic acid, propionic acid and/or butyric acid, in which
(a) an aqueous acetic acid mixture is crystallized by cooling to a temperature of 1° C. to 25° C. below the concentration-dependent liquidus temperature, and
(b) the crystalline phase formed is removed mechanically from the liquid phase, or
(c) the crystalline phase formed is partially remelted by gradual heating and the remaining crystalline phase is then removed mechanically.

It is clear to a person skilled in the art that, in principle, it is also possible to cool to temperatures of more than 25° C. below the concentration-dependent liquidus temperature. Physical reasons would not oppose this. For example, it may also be possible to cool to 50° C. or 100° C. below the liquidus temperature. As a result, the aqueous acetic acid mixtures would, however, completely freeze (crystallize), and so heating would then have to be carried out to 1° C. to 25° C. below the liquidus temperature again so that, according to the process disclosed herein, there is not only a crystalline phase (in which acetic acid is enriched), but also a liquid phase again (in which the secondary components to be removed are enriched), from which the crystalline phase can then be removed mechanically and/or by gradual melting.

The specified temperature range of 1° C. to 25° C. below the concentration-dependent liquidus temperature is preferred from an economic point of view, because additional energy would be consumed for any unnecessarily deeper cooling. The same would apply to the subsequently required heating to 1° C. to 25° C. below the concentration-dependent liquidus temperature.

In the context of step (c), removal can be effected, for example, mechanically and/or by complete melting after removal of the prior mother liquor. The preferred procedure is for the acetic acid to be completely crystallized, and then for the block formed to be partially remelted and for the mother liquor to be removed. The remaining block is then completely melted and crystallized again.

Step (c) of the process is advantageous if there are inclusions of the liquid phase in the crystalline phase and the resultant high viscosity means that the two phases cannot be mechanically separated from each other in a simple manner (e.g., by filtration). This may be the case for certain ratios of crystalline phase to liquid phase. As a rule of thumb, it is sufficient in such cases to remelt about 10% to 20% by weight of the originally formed crystalline phase by gradual heating in order to lower the viscosity such that mechanical removal (e.g., by filtration) is possible. Melting ranges that may deviate from this rule of thumb can be easily determined by a person skilled in the art by routine experiments.

Cooling can be effected in any manner, for example by placement into a freezer or by means of freezing mixtures (e.g. dry ice/acetone or ice/salt) or liquid nitrogen.

It is clear to a person skilled in the art that a fractional crystallization of acetic acid can also be achieved from the “opposite direction” by gradual cooling of an aqueous acetic acid mixture in the direction of the liquidus temperature. However, this is less advantageous from a practical and economic point of view.

The aqueous acetic acid mixture can contain at least 55%, 60%, 65%, 70%, 75%, 80%, 85%, 90% or 95% by weight of acetic acid.

In step (a), cooling can be carried out to 1° C.; 1.5° C.; 2° C.; 2.5° C., 3° C., 3.5° C.; 4° C.; 4.5° C., 5° C.; 5.5° C.; 6° C.; 6.5° C.; 7° C.; 7.5° C.; 8° C.; 9° C.; 10° C.; 12° C.; 15° C.; 18° C.; 20° C.; 22° C., 25° C. below the concentration-dependent liquidus temperature of the aqueous acetic acid mixture.

In step (a), cooling can be carried out for 2 min to 25 h. In step (a), cooling can be carried out in particular for 5 min, 10 min, 15 min, 20 min, 30 min, 40 min, 50 min, 60 min, 70 min, 80 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h or 24 h.

The mechanical method for removing the crystalline phase formed can be selected from one or more of the separation methods filtration, vacuum filtration, sedimentation, centrifugation and cyclonic separation.

Following step (b) or (c), steps (a) and (b) or steps (a) and (c) may be repeated at least once according to a step (d).

If the process is carried out repeatedly, step a) can be followed each time by carrying out only step b) or (alternatively) only step c). However, it is also possible that, after a predetermined number of repetitions, a switch is made from step b) to step c) (once only), or vice versa. Moreover, it is possible that, for example, execution of step a) is followed by assessment of the crystallization result, which is then followed by making a decision (as needed) as to whether step b) or step c) will be executed.

The invention will be described in detail in the following, wherein the disclosed specific embodiments of the invention, examples or results are intended for illustrative purposes only and are in no way to be interpreted as a limitation on the scope of the invention as defined in the accompanying claims.

### DETAILED DESCRIPTION

Unless otherwise specified, all the technical and scientific terms used herein have the same meaning as understood by a person skilled in the art in the technical field of the invention. A person skilled in the art may also make full reference to the introductory elucidations.

The use of definite or indefinite articles (“the”, “a”, “an”) is to be understood (especially in connection with the claims) such that at least one element or one component is to be encompassed thereby, unless otherwise indicated herein or unless the context clearly dictates otherwise.

The conjunction “or” is to be understood as an inclusive “or” and not as an exclusive “or”, i.e. as “and/or”, unless otherwise indicated herein or unless the context clearly dictates otherwise.

The use of terms such as “for example”, “e.g.”, “such as” or variations thereof is merely intended to better illustrate the invention and must in no way be interpreted as a limitation on the scope of the invention as defined in the accompanying claims.

All numerical values are understood to be “approximate” values (at least within the usual margins of error), irrespective of whether or not this is expressly indicated. Moreover, the recitation of ranges of values merely serves as a shortcut and, unless otherwise indicated herein, refers to each individual value falling within the range, even if said value is not individually recited.

It is known that acetic acid becomes solid at 16-17° C. This generally leads to problems in industrial handling. It has been found that a mixture containing acetic acid as the main component and water, formic acid, propionic acid, butyric acid and other constituents as the secondary components partially freezes at a temperature below the melting point of acetic acid. The secondary components interfere with the crystallization of acetic acid and thereby make purification thereof more difficult. The concentrations in the solid phase and the liquid phase are different. The crystalline phase is enriched with acetic acid and depleted of all the accompanying components water, formic acid, propionic acid and butyric acid. Considering the very different melting points and the chemical tendency of low-molecular-weight organic acids to form dimers by hydrogen bonds, the high enrichment of acetic acid in the presence of low boilers and high boilers by means of a crystallization technology is astonishing. It might be expected that substances having a relatively high melting point also preferentially precipitate during crystallization. Moreover, dimer formation works against crystallization. The more dimers, the lower the melting point and the higher the supercooling required for crystallization. Effective separation requires a precise procedure.

A process for purifying acetic acid by means of fractional crystallization from aqueous acetic acid mixtures containing formic acid, propionic acid and butyric acid as secondary constituents has not been described to date. Short-chain carboxylic acids form dimers with one another through hydrogen bonds between the carboxyl groups. The crystallization behaviour of a mixture containing short-chain carboxylic acids (e.g., that of a mixture containing formic acid, acetic acid, propionic acid and butyric acid) is unpredictable because of this dimer formation and because of, moreover, the dependence of the melting temperature or liquidus temperature of such mixtures on the concentrations of the individual carboxylic acid components. Against this background, failure of fractional crystallization for separation can be expected.

It is therefore all the more surprising that such fractional crystallization for separation is possible with the process described herein.

Advantageous and/or preferred embodiments of the process proposed here are the subject matter of the dependent claims.

In order to carry out the process disclosed herein, supercooling based on the melting point or liquidus point of acetic acid is required.

The degree of supercooling required for crystallization depends on the proportions of secondary components. The supercooling time influences the yield of the crystalline phase. It has additionally been found that, from a certain ratio of crystalline phase to liquid, there are inclusions of the liquid in the crystalline phase and the two phases cannot be separated from one another. The mixture must then be stirred mechanically or the temperature must be sufficiently increased to form a liquid flowable from the crystal phase.

Advantageously, the starting material used in the process disclosed herein is an acetic acid mixture which can contain 70% to 90% by weight of acetic acid, 1% to 5% by weight of formic acid, 1% to 5% by weight of water, 1% to 5% by weight of propionic acid and 1% to 5% by weight of butyric acid. It is clear to a person skilled in the art that the proportions of the components present in % by weight add up to 100% by weight. Specific individual values within the stated ranges for the components of the acetic acid mixture used are 75%, 80%, 85% by weight of acetic acid; 2%, 3%, 4% by weight of formic acid; 2%, 3%, 4% by weight of propionic acid and 2%, 3%, 4% by weight of butyric acid. It is self-evident that, depending on the starting material used, individual components can also be completely absent; a typical example that may be mentioned is an acetic acid mixture containing 90% by weight of acetic acid, 5% by weight of formic acid, 4% by weight of water and 1% by weight butyric acid.

This process disclosed herein has the advantages of dispensing with solvents, of working at low temperatures and of depleting both high boilers and low boilers in one process step.

The process disclosed herein will be further explained in the following examples. Said examples are intended to illustrate the invention only and must in no way be interpreted as a limitation on the scope of the invention as defined in the accompanying claims.

### Example 1

The degree of supercooling required for crystallization depends on the concentrations of acetic acid, water, formic acid, propionic acid and butyric acid, as follows. To this end, an acetic acid mixture is adjusted in temperature to −17° C. The temperature is measured in the acetic acid mixture. It initially falls exponentially and rises to a value T_{Sm} (melting temperature or liquidus temperature) upon onset of crystallization and then remains constant for a certain time t until it finally decreases again. Table 1 reports the temperatures T_{Sm} on the basis of some acetic acid mixtures:

TABLE 1

| | Acetic | Formic | | Propionic | Butyric | | |
|---|---|---|---|---|---|---|---|
| | acid | acid | Water | acid | acid | Other* | T_{Sm} in |
| Test | in % by weight | | | | | | ° C. |
| 1 | 100.0 | 0 | 0 | 0 | 0 | 0 | 16.3 |
| 2 | 96.0 | 4.0 | 0 | 0 | 0 | 0 | 13.1 |
| 3 | 92.0 | 8.0 | 0 | 0 | 0 | 0 | 9.4 |
| 4 | 88.0 | 12.0 | 0 | 0 | 0 | 0 | 5.6 |
| 5 | 84.0 | 16.0 | 0 | 0 | 0 | 0 | 2.7 |
| 6 | 80.0 | 20.0 | 0 | 0 | 0 | 0 | −0.8 |
| 7 | 86.1 | 4.4 | 3.7 | 4.2 | 0.4 | 1.2 | 4 |

*any other constituents, for examples aldehydes and ketones.

### Example 2

The acetic acid mixture from Test 7 in Example 1 is placed in a cold bath. The temperature is measured in the acetic acid mixture. The temperature of the cold bath is periodically lowered. At −1° C. in the acetic acid mixture, crystallization begins, and the temperature rises to 4° C. in the further course of the crystallization reaction.

### Example 3

Crystal growth is time-dependent. To investigate the dependency, an acetic acid mixture containing 86.1% by weight of acetic acid, 4.4% by weight of formic acid, 3.7% by weight of water, 4.2% by weight of propionic acid and 0.4% by weight of butyric acid is transferred to a beaker and placed into a cold bath at a temperature of −7° C. (melting temperature or liquidus temperature of this carboxylic acid mixture according to Table 1: 4° C.). Following onset of crystallization, which is discernible from a turbidity, 2 minutes are allowed to elapse and then filtration is carried out using a suction filter. The fractions are weighed. This test is repeated for different times t_{Kr} (crystallization time). Table 2 shows the results:

TABLE 2

| Test | t_{Kr} in min | Yield of crystal in % by weight |
|---|---|---|
| 1 | 2 | 9 |
| 2 | 10 | 15 |
| 3 | 90 | 31 |
| 4 | 1080 | 67 |

### Example 4

If the ratio of crystalline phase to liquid is too high, inclusions of the liquid will occur and it will no longer be able to flow. The mixture from Tests 1 and 2 from Example 3 can be filtered without further work-up by pouring the mixture out of the beaker. In the case of Tests 3 and 4 from Example 3, a non-flowable suspension is formed. It is therefore stirred mechanically with the aid of a glass rod before it is poured out.

### Example 5

The non-flowable suspension of Test 4 from Example 3 is heated from −7° C. to 2° C. This forms a flowable suspension which can be easily filtered by pouring it out.

### Example 6

The crystallization process is applied here in multiple stages. 60 g of an acetic acid mixture containing 86.1% by weight of acetic acid, 4.4% by weight of formic acid, 3.7% by weight of water, 4.2% by weight of propionic acid and 0.4% by weight of butyric acid are transferred to a beaker. In a crystallization stage 1, the mixture is held at −18° C. for 30 minutes. The beaker is placed onto a filter with the opening facing downwards and heated to 2° C. When droplet formation can no longer be seen at the outlet of the filter, the filtrate is weighed and a gas chromatogram is recorded. In a crystallization stage 2, heating is carried out from 2° C. to 7° C. The further course of action is as described above. In a crystallization stage 3, heating is carried out from 7° C. to 9° C. and the further course of action is as described above. The concentration in the crystalline phase is determined mathematically via the mass balance. Table 3 shows the results of this multi-stage crystallization:

TABLE 3

| | | Acetic | Formic | | Propionic | Butyric | |
|---|---|---|---|---|---|---|---|
| | | acid | acid | Water | acid | acid | Other |
| | Yield in g | in % by weight | | | | | |
| Acetic acid mixture | 60.0 | 86.1 | 4.4 | 3.7 | 4.2 | 0.4 | 1.2 |
| Crystal stage 1 | 32.6 | 89.0 | 3.4 | 3.1 | 3.1 | 0.3 | 1.1 |
| Crystal stage 2 | 22.2 | 91.4 | 2.7 | 2.3 | 2.3 | 0.2 | 1.1 |
| Crystal stage 3 | 15.4 | 93.7 | 2.1 | 1.7 | 1.9 | 0.1 | 0.5 |

It is clear to those skilled in the art in the technical field of the invention that the above-described representative embodiments and details of the invention are intended to illustrate the present invention only and that various alterations and modifications may be made without thereby departing from the scope of the invention as defined in the accompanying claims.

What is claimed is:

1. A process for enriching acetic acid from aqueous acetic acid mixtures containing at least 50% by weight of acetic acid and secondary constituents selected from the group consisting of formic acid, propionic acid and butyric acid, wherein the process comprises
(a) crystallizing an aqueous acetic acid mixture by cooling to a temperature of 1° C. to 25° C. below the concentration-dependent liquidus temperature, and
(b) removing mechanically the crystalline phase formed from the liquid phase, or
(c) partially remelting the crystalline phase formed by gradual heating and then removing mechanically the remaining crystalline phase.

2. The process according to claim 1, wherein the aqueous acetic acid mixture contains at least 55%, 60%, 65%, 70%, 75%, 80%, 85%, 90% or 95% by weight of acetic acid.

3. The process according to claim 1, wherein the aqueous acetic acid mixture contains 1% to 5% by weight of formic acid and/or 1% to 5% by weight of propionic acid and/or 1% to 5% by weight of butyric acid.

4. The process according to claim 1, wherein in step (a), cooling is carried out to 1° C.; 1.5° C.; 2° C.; 2.5° C., 3° C., 3.5° C.; 4° C.; 4.5° C., 5° C.; 5.5° C.; 6° C.; 6.5° C.; 7° C.; 7.5° C.; 8° C.; 9° C.; 10° C.; 12° C.; 15° C.; 18° C.; 20° C.; 22° C., 25° C. below the concentration-dependent liquidus temperature of the aqueous acetic acid mixture.

5. The process according to claim 1, wherein in step (a), cooling is carried out for 2 min to 25 h.

6. The process according to claim 5, wherein in step (a), cooling is carried out for 5 min, 10 min, 15 min, 20 min, 30 min, 40 min, 50 min, 60 min, 70 min, 80 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h or 24 h.

7. The process according to claim 1, wherein the mechanical method for removing the crystalline phase formed is selected from one or more of the separation methods filtration, vacuum filtration, sedimentation, centrifugation and cyclonic separation.

8. The process according to claim 1, wherein the process is carried out in multiple stages, wherein following step (b) or (c)
(d) steps (a) and (b) or steps (a) and (c) are repeated at least once.
